(19) United States
(12) Patent Application Publication
(10) Pub. No.: US 2009/0209750 A1
(43) Pub. Date: Aug. 20, 2009

(54) Organic compound synthesizer and method for synthesizing organic compounds

(75) Inventor: Hideaki Okayama, Tokyo (JP)

Correspondence Address: POSZ LAW GROUP, PLC, 12040 SOUTH LAKES DRIVE, SUITE 101, RESTON, VA, 20191, US

(73) Assignee: Oki Electric Industry Co., Ltd., Tokyo (JP)

(21) Appl. No.: 12/318,478

(22) Filed: Dec. 30, 2008

(30) Foreign Application Priority Data
Feb. 14, 2008 (JP) 2008-033521

(51) Int. Cl. C07H 21/04; B01J 19/24

(52) U.S. Cl. 536/25.3; 422/138

(57) ABSTRACT

An organic compound synthesizer for synthesizing organic compounds contains at least one type of polymerizable repeat unit and includes a substrate for organic compound synthesis. A liquid supply unit is configured to supply a reaction liquid containing compounds necessary for the synthesis of organic compounds and a reaction liquid containing a thermal acid generator for generating protons by heating. A substrate heater is configured to selectively heat a specific portion of said substrate for organic compound synthesis to thereby heat the reaction liquid containing a thermal acid generator.

### CROSS REFERENCE TO RELATED APPLICATION

The present application is related to and claims priority from Japanese Patent Application No. 2008-033521, filed on Feb. 14, 2008, the contents of which are incorporated herein by reference.

### TECHNICAL FIELD

The present invention relates generally to an organic compound synthesizer, and specifically to an organic compound synthesizer provided for semiconductor manufacturing devices and the like and a method for synthesizing organic compounds.

### BACKGROUND

Methods of using a chip having a substrate on which deoxyribonucleic acid, or DNA, as biological material is arranged have been researched and put into practical use for producing medicines tailored to the needs of specific individuals.

A DNA chip has DNA segments having diverse sequences spot-arranged on a substrate. Two methods for making a DNA chip have been proposed. One method involves synthesizing as many DNA segments as the number of spots on the substrate in advance and then arranging the synthesized DNA segments on the substrate by an ink jet method. The other method involves synthesizing DNA by linking nucleic acids on a substrate.

The method of synthesizing DNA by linking nucleic acids on a substrate is superior in that DNA of any sequence can directly be synthesized on a substrate using only materials corresponding to four bases. Such a method also allows patterning DNA on a substrate by a method used for optical lithography or an ink jet method.

The method of synthesizing DNA by linking nucleic acids on a substrate is carried out as follows. A protecting group is formed on a substrate via a predetermined linker. This protecting group reacts with light or acid and is removed from the linker. As a result, the edge portion of the linker having a hydrogen atom is exposed. Then, a base with a protecting group is reacted with the edge portion to link a portion of the base having an isopropyl (iPr) group to the edge portion of the linker. The protecting group is removed by irradiating light or supplying an acid to a site where a new base should be linked. Then, a new base is linked to the portion from which the protecting group is removed. Any base sequence can be formed by repeating these processes, thereby resulting in the formation of a DNA chip.

In the case of carrying out a genetic diagnosis using a DNA chip formed by the aforementioned processes, cDNA (complementary DNA) is first obtained from mRNA (messenger RNA) synthesized for a gene that is expressed in a specimen. Then, the cDNA is added to, for example, a fluorescent label that can provide signals necessary for detection. The specimen is added to a DNA chip. As a result, only cDNA that matches with the sequence on the DNA chip can bind to a specific portion of base sequence. A portion in which DNA on the DNA chip binds to cDNA in the specimen can be detected by detecting fluorescence emitted from the fluorescent label, whereby diseases can be diagnosed. It is also possible to detect a difference in a gene by dividing the gene into fragments and carrying out the same processes.

It is possible to determine which DNA sequence should be synthesized at which position on a substrate by specifying a position or spotting to which light is irradiated or an acid is supplied. It is conventional in the art that a light irradiation pattern on a substrate is formed by using a glass mask such as that used for manufacturing a semiconductor integrated circuit. The method of using a glass mask is suitable for mass-producing the same product yet has a problem that it is not suitable for readily adjusting to a pattern change.

Methods for adjusting to a pattern change have been proposed using a micromachine mirror or liquid crystal used for displays. However, since these methods use a projection optical system like a glass mask, there is a limit to how much a device can be miniaturized.

Optical systems using laser scanning or optical fibers have also been proposed. However, these systems are not intended for devices of practical use. Methods using a glass mask are excellent in the uniformity of chemical substances in a spot. However, other methods have a problem in the uniformity of chemical substances.

The problems associated with the aforementioned the ink jet method are that precision is required for positioning each spotting and that DNA sections tend to become uneven due to irregularities at the time of drying.

### SUMMARY

In view of the above, various exemplary embodiments of a novel and improved organic compound synthesizer and a method of synthesizing organic compounds that are readily adjustable in response to the change of a sequence pattern of organic compounds will be described. These embodiments are superior to conventional synthesizers and methods as a result of the uniformity of the resulting synthesized organic compounds and as they enable miniaturization.

According to one exemplary embodiment, an organic compound synthesizer for synthesizing organic compounds contains at least one type of polymerizable repeat unit. The organic compound synthesizer comprises a substrate for organic compound synthesis. A liquid supply unit is configured to supply a reaction liquid containing compounds necessary for the synthesis of organic compounds and a reaction liquid containing a thermal acid generator for generating protons by heating. A substrate heater is configured to selectively heat a specific portion of said substrate for organic compound synthesis to thereby heat the reaction liquid containing a thermal acid generator.

The organic compound synthesizer according to the present invention allows adjustments to be readily made in response to the change of a sequence pattern of organic compounds, is superior in the uniformity of synthesized organic compounds and allows for miniaturization.

### BRIEF DESCRIPTION OF THE DRAWINGS

FIG. 1A is a plan view illustrating a substrate for organic compound synthesis in an organic compound synthesizer according to a first embodiment.

FIG. 1B is an explanatory view illustrating a substrate heating unit in the organic compound synthesizer according to the first embodiment.

FIG. 1C is a sectional view illustrating the organic compound synthesizer according to the first embodiment.

FIG. 2 is an explanatory view illustrating the operation of the organic compound synthesizer according to the first embodiment using DNA synthesis as an example.

FIG. 3 is an explanatory view illustrating a first variation of the organic compound synthesizer according to the first embodiment.

FIG. 4A is a sectional view illustrating an organic compound synthesizer according to a second embodiment.

FIG. 4B is an explanatory view illustrating a substrate heating unit in the organic compound synthesizer according to the second embodiment.

### DETAILED DESCRIPTION

The following is a detailed description of the preferred embodiment of the present invention with reference to drawings. In the specification and drawings, identical reference numerals are used to designate corresponding constituent elements that are substantially common to drawings, thus avoiding redundant explanation.

Moreover, the instant disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued. It is further understood that the use of relational terms such as first and second, and the like, if any, are used solely to distinguish one from another entity, item, or action without necessarily requiring or implying any actual such relationship or order between such entities, items or actions.

It is noted that some embodiments may include a plurality of processes or steps, which can be performed in any order, unless expressly and necessarily limited to a particular order; i.e., processes or steps that are not so limited may be performed in any order.

As described above, the organic compound synthesizer according to the various exemplary embodiments is a device for synthesizing organic compounds containing one or more repeat units. Organic compounds synthesized by this device include biological macromolecular compounds such as nucleic acids (e.g., DNA and RNA), proteins and regular xenobiotic macromolecular compounds (e.g., polymers and oligomers). In the case of nucleic acids such as DNA and RNA, a repeat unit consists of four types of bases (i.e., A, T, G and C). In the case of proteins, a repeat unit consists of various amino acids. In the case of regular macromolecular compounds, a monomer is the repeat unit.

As will now be described, organic compound synthesizers of first and second exemplary embodiments are exemplified by a DNA array synthesizer for synthesizing DNA arranged in arrays.

### First Embodiment

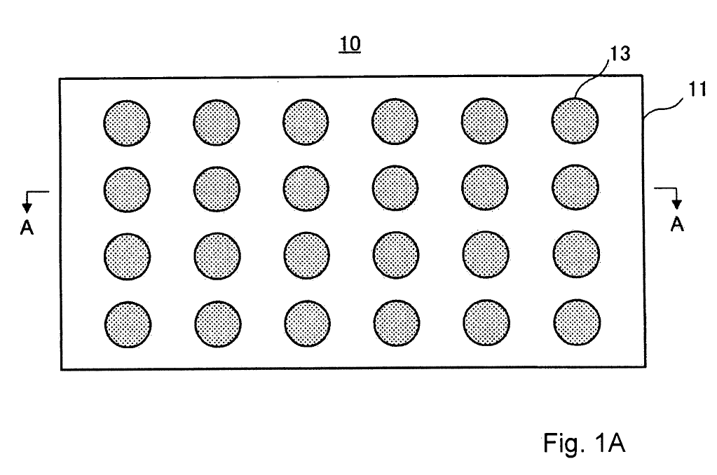

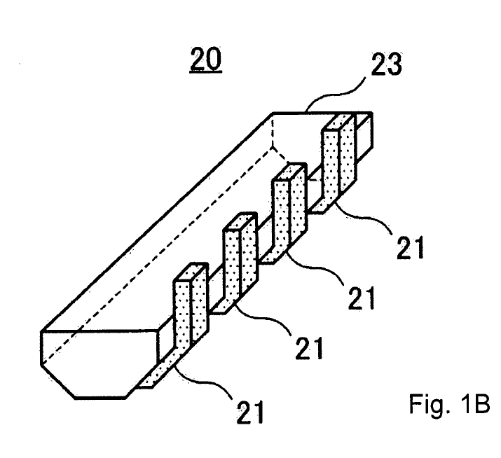

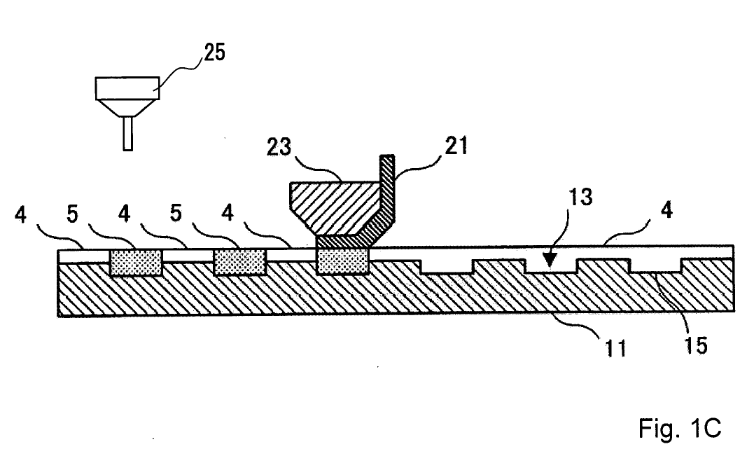

The following is a detailed description of the organic compound synthesizer of the first embodiment. FIG. 1A is a plan view illustrating a substrate for organic compound synthesis in the organic compound synthesizer according to the present embodiment. FIG. 1B is an explanatory view illustrating a substrate heating unit in the organic compound synthesizer according to the present embodiment. FIG. 1C is a sectional view illustrating the organic compound synthesizer according to the present embodiment.

The organic compound synthesizer according to the present embodiment primarily includes a substrate for organic compound synthesis 10 and a substrate heating unit 20.

As shown in FIG. 1A, the substrate for organic compound synthesis 10 includes a base substrate 14 and sections 13.

The base substrate 11 can be made of metal (e.g., aluminum), glass or plastic. The material of the base substrate 11 is determined in accordance with the reaction conditions of organic compounds. For example, it is preferable to use a substrate superior in solvent resistance in the organic compound synthesizer according to the present embodiment because acid and alkali are used in the reaction. If the reaction is induced by irradiating light, it is preferred to use a substrate that allows light within a specified wavelength to pass through.

A plurality of sections 13 is formed on the base substrate 11 horizontally and vertically. In other words, the sections 13 are arranged in a matrix (rows and columns) in a plane. Each section 13 may be any size. For example, one section 13 may have a size of about several tens of micrometers (μm).

In FIG. 1A, the shape of each of the sections 13 is substantially circular. However, the shape is not restricted to a circle and may alternatively be a substantially elliptic shape, a polygonal shape such as a substantially square shape, or a substantially pentagonal shape. In FIG. 1A, the sections 13 are formed by 4 columns and 6 rows on the base substrate 11. However, the numbers of rows and columns are not limited to this example. The uniformity of synthesized organic compounds becomes high by using a substantially circular shape for the sections 13, however.

As shown in FIG. 1B, substrate heating unit, or more generally substrate heater, 20 includes a thermal head 23 on which electric heaters 21 are arranged in an array. In other words, the electric heaters 21 are arranged linearly in a row-column format. The electric heaters 21 provided on the thermal head 23 may be those that allow locally applying heat to an area within a range of several tens of microns. The number of electric heaters 21 provided on the thermal head 23 is preferably at least the same as the number of columns or rows of the sections 13 on the substrate for organic compound synthesis 10, for example. The plurality of electric heaters 21 can be turned on and off independently. It is therefore possible to select sections 13 to be heated by controlling the power sources of electric heaters 21.

It is also possible to use a thermal head used for a printer as the thermal head 23 for the substrate heating unit 20 according to the present embodiment.

FIG. 1C is a sectional view of the substrate for organic compound synthesis 10 taken through section line A-A in FIG. 1A. FIG. 1C also illustrates the substrate heating 20.

The sections 13 of the substrate for organic compound synthesis 10 according to the present embodiment may be flat or may be provided with grooves 15 as shown in FIG. 1C. The grooves 15 of the sections 13 help to prevent organic compounds synthesized in the grooves 15 and the thermal head 23 from coming into contact with each other.

As shown in FIG. 1C, a reaction liquid 4 containing a thermal acid generator (TAG), which generates protons or acid by a rise in temperature, is supplied to the substrate for organic compound synthesis 10 by a liquid supply unit, shown generally at 25. A conventionally well-known thermal acid generator may be used. Included are onium salts such as iodonium salts, sulfonium salts, phosphonium salts and diazonium salts. Of these, it is preferable to use sulfonium salts that have an SbF₆-ion or a PF₆-ion as a counter ion. The reaction liquid 4 containing a thermal acid generator may be fluid or gel.

The reaction liquid 4 containing a thermal acid generator may further contain compounds necessary for synthesizing organic compounds. However, it is preferred that such compounds are separately supplied to the substrate for organic compound synthesis 10.

The thermal head 23 of the substrate heating unit 20 moves from one end of the substrate for organic compound synthesis 10 to the opposite end with time above the reaction liquid 4 containing the thermal acid generator after it is supplied to the substrate for organic compound synthesis 10. The electric heater 21 of the thermal head 23 generates heat intermittently at a desired position while moving in order to apply heat to the reaction liquid 4 containing a thermal acid generator. The heated thermal acid generator generates protons in order to form an acid region 5, in which liquid is acidic, in the section 13 heated by the thermal head 23. In the acid region 5, the chemical reaction for synthesizing organic compounds advances in order to produce a desired organic compound.

In the case that a reaction liquid containing compounds necessary for synthesizing organic compounds is supplied separately from a reaction liquid containing a thermal acid generator, the reaction liquid 4 containing a thermal acid generator is first removed after heating the reaction liquid 4 containing a thermal acid generator by the thermal head 23 to form the acid region 5, and then a reaction liquid containing compounds necessary for synthesizing organic compounds is supplied. As a result, a desired organic compound can be synthesized. Reaction liquids may be supplied to the same bath. Or, different baths may be used for each reaction liquid. If each reaction liquid is supplied to a different bath, the operation may be automated using a carrier system. However, for ease of illustration, and for purposes of discussion, a single liquid supply unit for supplying both reaction liquids is shown in FIG. 1C.

In the case that compounds necessary for synthesizing organic compounds are contained in the reaction liquid 4 containing a thermal acid generator, the compounds contained in the reaction liquid 4 bring about chemical reaction in the acid region 5 formed by the heating of the thermal head 23 in order to synthesize a desired compound.

In FIG. 1C and FIG. 1B, the lower edge of the thermal head 23 is flat. However, it is preferred to make the lower edge of the thermal head 23 round to minimize the disturbance of each portion of reaction liquid supplied to the substrate for organic compound synthesis 10.

Operation

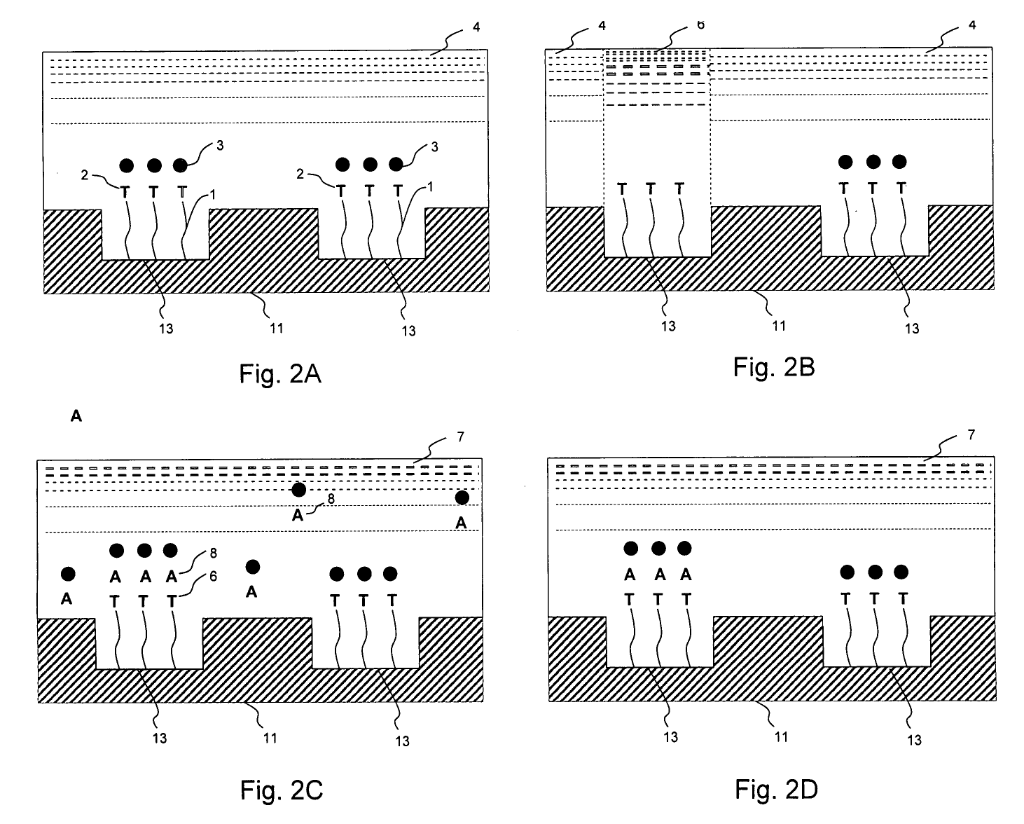

The following is a detailed description of the organic compound synthesizer according to the present embodiment with reference to FIGS. 2A-2D, which illustrate the operation of the organic compound synthesizer according to the present embodiment exemplified by DNA synthesis. In this case, the aforementioned polymerizable repeat units correspond to four types of bases: adenine (A), thymine (T), guanine (G) and cytosine (C).

As shown in FIG. 2A, a base (thymine) 2 having a protecting group 3 is formed in a section 13 of the substrate for organic compound synthesis 10 according to the present embodiment via a linker 1. The reaction liquid 4 containing a thermal acid generator is supplied to the substrate for organic compound synthesis 10 by a liquid supply unit 25 shown in FIG. 1C. The reaction liquid 4 containing a thermal acid generator is heated by the thermal head 23 in order to form the acid region 5 in a heated location. The protecting group 3 is removed from the base 2 by generated acid (protons) to produce a base 6 capable of reacting (FIG. 2B).

As shown in FIG. 2C, the reaction liquid 4 containing a thermal acid generator is removed form the substrate for organic compound synthesis 10, and then an amidite solution 7, which is a reaction liquid containing a desired base 8 (adenine having a protecting group 3 in the example shown in FIG. 2C), is supplied. As a result, the base 6, which has lost the protecting group 3 and become capable of reacting, reacts with the base 8 having a protecting group 3 to elongate DNA. Here, DNA is not elongated in the sections 13 where no heat was applied by the thermal head 23 because the bases 2 in those sections 13 still have protecting groups 3. Thus, as shown in FIG. 2D, DNA elongated only with one base is produced.

After washing the substrate for organic compound synthesis 10 with cleaning fluid, DNA having a desired base sequence can be synthesized on the substrate for organic compound synthesis 10 by repeating the processes of FIG. 2A-FIG. 2D by substituting solution containing a base to be added.

### First Alternative Embodiment

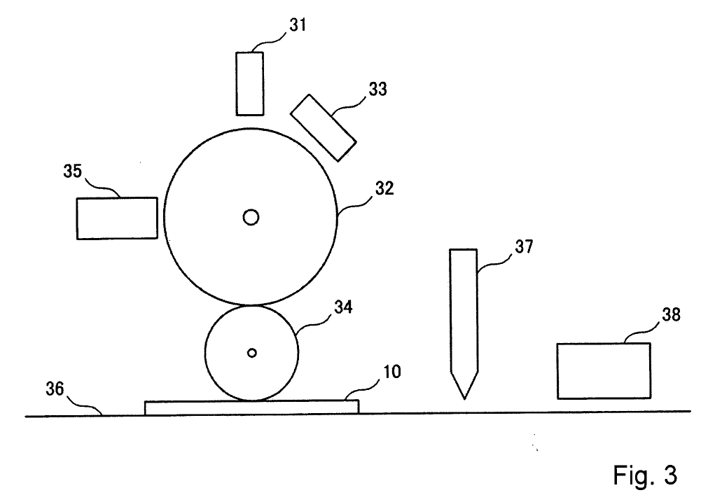

FIG. 3 is an illustrates a the first alternate embodiment of the above described organic compound synthesizer.

In the organic compound synthesizer according to the first embodiment of the present invention, the substrate for organic compound synthesis 10 is directly heated by the substrate heating unit 20. In the organic compound synthesizer according to this alternate embodiment, the substrate for organic compound synthesis 10 is not directly heated by the substrate heating unit 20. The organic compound synthesizer according to this alternate embodiment is effective for synthesizing protein or the like that are prone to being degenerated by heating.

As shown in FIG. 3, the organic compound synthesizer according to this alternate embodiment primarily includes a substrate for organic compound synthesis 10, a reaction liquid feeder 31 for supplying a reaction liquid containing a thermal acid generator, a drum 32, a thermal head 33, a transfer drum 34, a cleaner 35, a carrier device 36, a reaction device 37 and a cleaning device 38.

The substrate for organic compound synthesis 10 has the same configuration as that of the substrate for organic compound synthesis 10 according to the first embodiment and therefore will not be described again.

The reaction liquid feeder 31 is configured to supply a reaction liquid containing a thermal acid generator to the drum 32. The reaction liquid feeder 31 may be a spray type device, a roller type device or a syringe type device, for example.

The thermal head 33 is configured to heat a predetermined portion of the reaction liquid containing the thermal acid generator placed on the drum 32. As the thermal head 33, a unit or module similar to the previous thermal head 23 which is directed to the first embodiment may be practical. Further, a plural of the thermal heads 33 can be equipped with the organic compound synthesizer with respect to the alternative embodiment.

The transfer drum 34 is configured to transfer a reaction liquid containing a thermal acid generator, which is on the drum 32 and heated at a desired place, to the substrate for organic compound synthesis 10. It is possible to directly transfer a reaction liquid to the substrate for organic compound synthesis 10 from the drum 32 without using the transfer drum 34. Nevertheless, the transfer drum 34 can enhance the transferability of a reaction liquid to the substrate for organic compound synthesis 10.

The cleaner 35 is configured to clean and bring back to the initial state the drum 32 after the reaction liquid containing a thermal acid generator heated at a desired place is transferred to the transfer drum 34 or the substrate for organic compound synthesis 10. The cleaner 35 may be a blade type device or a roller type device. Or, it may be a scraper used for a printer or a cleaning device using an antacid. In FIG. 3, the cleaner 35 is provided for the drum 32. However, a cleaner 35 may also be provided for the transfer drum 34 if necessary.

The reaction device 37 is configured to supply a reaction reagent necessary for synthetic reaction to the substrate for organic compound synthesis 10 transported by the carrier device 36 in order to advance synthetic reaction. The reaction device 37 may be used for coating the substrate for organic compound synthesis 10 with a reaction liquid containing compounds necessary for synthesis or may be a bath holding a reaction liquid containing compounds necessary for synthesis. It is also possible to provide a plurality of reaction devices 37 depending on the type and property of reagent necessary for reaction.

Although FIG. 3 shows only one reaction device 37, two or more reaction devices may be provided in the organic compound synthesizer according to the present alternate embodiment. Merely by way of example, in the case of DNA synthesis, four reaction devices 37 may be provided that correspond to four types of base: adenine (A), thymine (T), guanine (G) and cytosine (C). In the case of a synthesizing copolymer of n components having n types of repeat units, n reaction devices 37 may be provided.

The cleaning device 38 is configured to remove excess reaction liquid and unreacted materials from the substrate for organic compound synthesis 10. At the time of removing unreacted materials, the substrate for organic compound synthesis 10 may be submerged in a bath holding chemicals used for removing unreacted materials.

The substrate for organic compound synthesis 10 provided on the carrier device 36 moves back and forth between the transfer drum 34, the reaction device 37 and the cleaning device 38. As a result, a compound is synthesized on the substrate for organic compound synthesis 10 by layering.

The configuration of the reaction liquid feeder 31, drum 32, thermal head 33, transfer drum 34, cleaner 35 and carrier device 36 of the organic compound synthesizer are not limited to that as shown in FIG. 3. The configuration can be changed in any appropriate manner.

### Second Embodiment

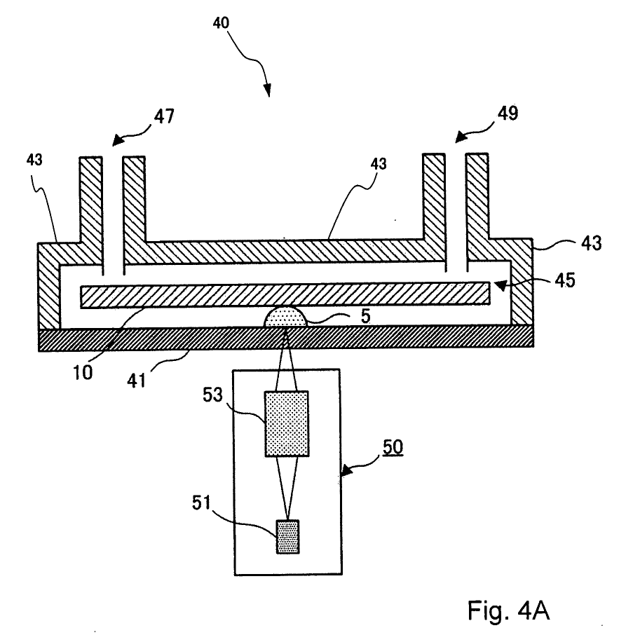

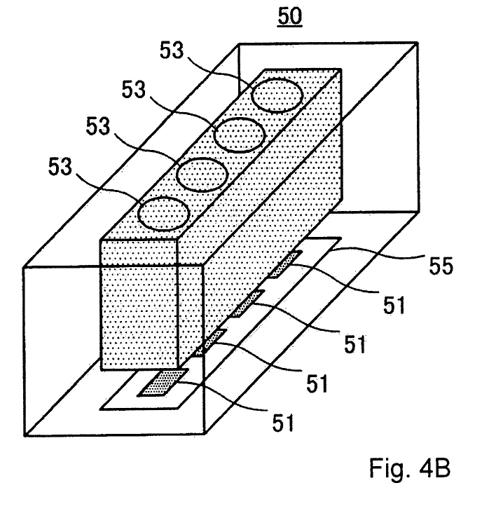

The following is a detailed description of the organic compound synthesizer according to a second exemplary embodiment with reference to FIG. 4A and FIG. 4B. FIG. 4A is a sectional view illustrating the organic compound synthesizer according to the second embodiment. FIG. 4B is an explanatory view illustrating the substrate heating unit of the organic compound synthesizer of FIG. 4A.

As shown in FIG. 4A, the organic compound synthesizer according to the second exemplary embodiment primarily includes a reaction container 40 for placing a substrate for organic compound synthesis 10 therein and an optical illuminator 50, which is the substrate heating unit.

The reaction container 40 for placing the substrate for organic compound synthesis 10 including both a top surface and a bottom surface, includes a heat production substrate 41, which includes a bottom surface and a top surface positioned a predetermined distance apart from a bottom surface of the substrate for organic compound synthesis 10, and a cover substrate 43, which covers one face of the heat production substrate 41 (i.e., the face on the side of the substrate for organic compound synthesis 10 in the present embodiment) in such a way as to enclose the substrate for organic compound synthesis 10 and is arranged in such a way as to have a specific space between itself and the substrate for organic compound synthesis 10.

The space created among the heat production substrate 41, the cover substrate 43 and the substrate for organic compound synthesis 10 is defined as a reaction liquid holding section 45 for holding various reaction liquids used for synthesizing organic compounds.

The cover substrate 43 is provided with two through holes. One is used as a reaction liquid inlet 47 and the other as a reaction liquid outlet 49. Various liquids used for synthesizing organic compounds are introduced from a liquid supply unit such as that shown at 25 in FIG. 1C to the reaction liquid inlet 47, pass through the reaction liquid holding section 45 and are discharged from the reaction liquid outlet 49. Thus, a passage for reaction liquids is formed in the reaction container 40 according to the second embodiment as follows: the reaction liquid inlet 47→the reaction liquid holding section 45→the reaction liquid outlet 49. The specific structure of the reaction container 40 is represented by a microfluid structure, which is formed by digging a groove on a substrate.

The substrate for organic compound synthesis 10 has the same configuration as that of the substrate for organic compound synthesis 10 according to the first embodiment and has substantially the same effect. Therefore, for the sake of brevity, a detailed explanation of the substrate 10 in the second embodiment is omitted.

The heat production substrate 41 is capable of absorbing light emitted from an optical illuminator 50 such as described below and converting it to heat. The heat production substrate 41 may be formed by evaporating a thin molybdenum (Mo) film on quartz glass. A reaction liquid can efficiently be heated by production heat by placing the side of the evaporated thin molybdenum film toward the side of the substrate for organic compound synthesis 10.

The optical illuminator 50, which is the substrate heating unit, or heater, of the organic compound synthesizer according to the second embodiment, includes a plurality of light sources 51 and emits light toward a reaction liquid (i.e., a reaction liquid containing a thermal acid generator) held in the reaction liquid holding section 45 of the reaction container 40. Thus, the irradiation of light to a reaction liquid held in the reaction liquid holding section 45 of the reaction container 40 from the light source 51 of the optical illuminator 50 allows for photochemical reaction having a spatial distribution for the chemical reaction system formed by the reaction container 40 containing the substrate for organic compound synthesis 10 and the optical illuminator 50.

As shown in FIG. 4A and FIG. 4B, the optical illuminator 50 according to the second embodiment primarily includes multiple light sources 51 arranged on a light source array substrate 55 in an array (linearly) and a rod lens array in which rod lenses 53 are arranged in an array (linearly). Although the multiple light sources 51 according to the second embodiment are arranged in one row in an array as described above. It is not necessary for them to be arranged in an array. Nor is the number of rows of the light sources 51 limited to one. It may be appropriately changed depending on the size of the substrate for organic compound synthesis 10 and a light irradiation pattern formed on the substrate for organic compound synthesis 10. Moreover, the number of the light sources 51 can properly be selected depending on the size of the substrate for organic compound synthesis 10 and a light irradiation pattern formed on the substrate for organic compound synthesis 10.

As the light source 51, an optical element using a semiconductor may be used, for example. Such an optical element includes a light emitting diode and a semiconductor laser. As for emission wavelengths, any desired emission wavelength may be used. However, it is preferred to use wavelengths in the near infrared range or infrared range.

In the case of using a light emitting diode as the light source 51, a pitch of light emitting diodes (i.e., an interval of adjacent light emitting diodes) may be about 20 μm and the focal distance of a rod lens 53 about 4 mm.

The use of a light emitting diode allows the use of a lens with a shorter focal distance compared with that of a conventional instrument using a UV lamp and a laser, which leads to a reduction in the distance between the reaction container 40 containing the substrate for organic compound synthesis 10 and the optical illuminator 50. As a result, the entire size of the organic compound synthesizer can therefore be reduced.

The rod lens 53 is one example of a light condensing lens for converging light emitted from the light source 51 and adjusting the converged light to a desired focal position. In the second embodiment, a focal position is adjusted by the rod lens 53 such that light emitted from the light source 51 is converged to the thin molybdenum film of the heat production substrate 41.

In the second embodiment, the rod lenses 53 are arranged in one row in an array. However, it is not necessary for them to be arranged in an array. A zigzag arrangement is also possible. Moreover, the arrangement and number of the rod lenses 53 are not particularly limited as are the light sources 51.

The organic compound synthesizer according to the second embodiment has a moving mechanism (not shown) in order to move the optical illuminator 50 with respect to the reaction container 40. The moving mechanism can be configured in such a way as to move, for example, the optical illuminator 50 in the direction perpendicular to the axial direction of the array of the light sources 51, that is, in the direction in parallel to the substrate for organic compound synthesis 10. The direction of the relative movement of the optical illuminator 50 is not limited to the aforementioned example. Movement may be a diagonal direction of the substrate for organic compound synthesis 10. Or, it can be configured such that the optical illuminator 50 may relatively be moved along the substrate for organic compound synthesis 10 while making a round trip.

Thus, a spatial pattern can be formed at a position where an acid range 5 is created on the substrate for organic compound synthesis 10 by temporally and spatially turning on or off light irradiation from the light source 51 while moving the optical illuminator 50 with respect to the substrate for organic compound synthesis 10 so as to selectively irradiate light to a desired place where acid is produced from a thermal acid generator.

The formation of a spatial pattern (i.e., a light irradiation pattern) at a position where an acid range 5 is created in this manner enables the spatial pattern of light irradiation (i.e., a spatial pattern of positions where an acid range 5 is created) to be easily changed merely by controlling the timing of turning on and off light irradiation from the light source 51 at the time of relative motion of the optical illuminator 50.

In the second embodiment, a spatial pattern is formed at a position where an acid range 5 is created on the substrate for organic compound synthesis 10 by relatively moving the optical illuminator 50 with respect to the substrate for organic compound synthesis 10 in the direction perpendicular to the axis of the array, wherein the optical illuminator 50 has a structure of the light sources 51 and rod lenses 53 arranged in an array. The formation of the aforementioned spatial pattern allows precise irradiation of light to a desired place only by relatively moving the optical illuminator 50 with respect to the substrate for organic compound synthesis 10, which leads to enhancing the uniformity of chemical materials and forming a spatial pattern at high speed.

In the case of synthesizing organic compounds using the substrate for organic compound synthesis 10 according to the first exemplary embodiment and the optical illuminator 50 according to the present exemplary embodiment, it is preferable to add nanoparticles and compounds that absorb light emitted from the optical illuminator 50 and convert it to heat to the reaction liquid 4 containing a thermal acid generator. By adding the aforementioned materials to the reaction liquid 4 containing a thermal acid generator, energy of light emitted from the optical illuminator 50 can efficiently be converted to heat. In this case, the heat production substrate 41 can be omitted.

The aforementioned compounds that absorb light and produce heat include dyes and pigments that can absorb light in the range of not shorter than 700 nm in wavelength (preferably in the range of 750 nm to 1200 nm) with high efficiency.

Such dyes include azo dyes, metal complex azo dyes, pyrazolone azo dyes, naphthoquinone dyes, anthoraquinone dyes, phthalocyanine dyes, carbonium dyes, quinone imine dyes, methine dyes, cyanine dyes, squaririum dyes, pyrylium salts and metal thyolate complexes. The aforementioned pigments include insoluble azo pigments, azo lake pigments, condensed azo pigments, chelate azo pigments, phthalocyanine pigments, anthoraquinone type pigments, perylene and perynone type pigments, thioindigo type pigments, quinacridone type pigments, dioxazine type pigments, isoindolinone type pigment, quinophthalone type pigments, color lake pigments, azine pigments, nitroso pigments, nitro pigments, natural pigments, fluorescent pigments, inorganic pigments and carbon black.

Operation

The light emitted from the light source 51 is absorbed by the heat production substrate 41. The heat production substrate 41 produces heat depending on the energy of the light emitted from the light source 51. The heat thus produced heats a reaction liquid containing a thermal acid generator held in the reaction liquid holding section 45 to produce acid (protons), thus creating an acid region 5. The organic compound synthesizer according to the present embodiment allows synthesizing organic compounds using acid thus to be produced. Here, we omit describing the synthetic reaction of organic compounds using acid in detail because it is essentially the same as the reaction shown in FIGS. 2A-2D.

Examples of Usable Reagents

The following is a more specific description of the methods for synthesizing DNA in accordance with the first and second embodiments by showing some examples of usable reagents.

The synthetic chemistry of DNA has continuously been developed and includes a variety of methods. The most commonly used method is referred to as the 4-step method using phosphoramide. The organic compound synthesizer according to the present embodiments supplies a thermal acid generator that produces acid (protons) by heating unlike the conventional 4-step method in which acid (TCA) is supplied. The following is a detailed description of the method for synthesizing DNA according to the disclosed embodiments.

First, 2′-deoxynucleoside with a 5′-dimethoxytrityl group is fixed as a protecting group to a linker provided in sections 13 formed on the surface of the base substrate 11 made of glass. A conventional method may be used for fixing the base to a linker. The base substrate 11 is formed by either a glass-system substrate or a polystyrene substrate.

As described above, any thermal acid generator (TAG) may be used as a photogenerated acid (PGA). For example, thermal acid generators include SbF₆ type sulfonium salts and PF₆ type sulfonium salts that are often used for resist materials.

In the case of producing heat by using optical absorption, the following can be used, for example: dyes such as azo dyes, metal complex azo dyes, pyrazolone azo dyes, naphthoquinone dyes, anthoraquinone dyes, phthalocyanine dyes, carbonium dyes, quinone imine dyes, methine dyes, cyanine dyes, squaririum dyes, pyrylium salts and metal thyolate complexes; and pigments such as insoluble azo pigments, azo lake pigments, condensed azo pigments, chelate azo pigments, phthalocyanine pigments, anthoraquinone type pigments, perylene and perynone type pigments, thioindigo type pigments, quinacridone type pigments, dioxazine type pigments, isoindolinone type pigment, quinophthalone type pigments, color lake pigments, azine pigments, nitroso pigments, nitro pigments, natural pigments, fluorescent pigments, inorganic pigments and carbon black.

After washing with acetonitrile (CH₃CN) and dichloromethane (CH₂Cl₂), DNA is eleongated in a tetrazole/CH₃CN solution.

After the elongation reaction, CH₃CN is used again for washing. Then, a mixture of acetic anhydride/lutidine/tetrahydrofurane (THF) and N-methylimidazol/THF is used for capping. After washing, I₂/THF/pyridine/H₂O are used for oxidation. Thus, washing, capping and washing make one cycle.

Hence, the organic compound synthesizer according to the exemplary embodiments enables a compound chip to be produced on which any organic compound can be arranged. It is therefore possible to realize a small-sized and high-speed organic compound synthesizer having the same advantages as printers.

Moreover, thermal heads developed for printers and communication, compound semiconductor LED and semiconductor laser can be used as a heat source, resulting in a reduction in the costs of manufacturing organic compound synthesizers.

This disclosure is intended to explain how to fashion and use various embodiments in accordance with the invention rather than to limit the true, intended, and fair scope and spirit thereof. The invention is defined solely by the appended claims, as they may be amended during the pendency of this application for patent, and all equivalents thereof. The foregoing description is not intended to be exhaustive or to limit the invention to the precise form disclosed. Modifications or variations are possible in light of the above teachings. The embodiments were chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention as determined by the appended claims, as may be amended during the pendency of this application for patent, and all equivalents thereof, when interpreted in accordance with the breadth to which they are fairly, legally, and equitably entitled.

For example, DNA was used as a desired organic compound in the aforementioned embodiments. However, it is also possible to synthesize proteins and regular macromolecular compounds as desired organic compounds in the same manner.

What is claimed is:

1. An organic compound synthesizer for synthesizing organic compounds containing at least one type of polymerizable repeat unit, said synthesizer comprising:
a substrate for organic compound synthesis;
a liquid supply unit configured to supply a reaction liquid containing compounds necessary for said synthesis of organic compounds and a reaction liquid containing a thermal acid generator for generating protons by heating to said substrate; and
a substrate heater configured to selectively heat a specific portion of said substrate for organic compound synthesis to thereby heat said reaction liquid containing a thermal acid generator.

2. The organic compound synthesizer according to claim 1, wherein said substrate heater is a thermal head provided with a plurality of electric heaters disposed in an array.

3. The organic compound synthesizer according to claim 1, wherein said substrate heater includes an optical illuminator configured to emit light of a specified wavelength for heating said reaction liquid containing a thermal acid generator.

4. The organic compound synthesizer according to claim 1, wherein said substrate for organic compound synthesis includes grooves formed in a matrix for holding said reaction liquid containing compounds necessary for synthesizing organic compounds and said reaction liquid containing a thermal acid generator.

5. The organic compound synthesizer according to claim 1, wherein said reaction liquid containing compounds necessary for synthesizing organic compounds is an amidite reagent.

6. The organic compound synthesizer according to claim 1, wherein said liquid supply unit includes a reaction liquid feeder configured to supply a reaction liquid containing a thermal acid generator to said substrate for organic compound synthesis and a reaction device configured to supply a reaction reagent necessary for synthetic reaction to said substrate for organic compound synthesis.

7. The organic compound synthesizer according to claim 1, wherein said substrate for organic compound synthesis includes sections formed in a matrix on which organic compounds are synthesized, and wherein the shape of each of the sections is substantially circular.

8. The organic compound synthesizer according to claim 1, wherein the substrate for organic compound synthesis is directly heated by the substrate heater.

9. The organic compound synthesizer according to claim 2, wherein
said substrate for organic compound synthesis includes sections formed in columns and rows on which organic compounds are synthesized, and
the number of electric heaters provided on the thermal head is at least the same as the number of columns or rows of the sections.

10. The organic compound synthesizer according to claim 2, wherein the plurality of electric heaters are turned on and off independently.

11. The organic compound synthesizer according to claim 2, wherein a lower edge of the thermal head is rounded.

12. The organic compound synthesizer according to claim 3, further comprising a heat production substrate positioned apart from said substrate for organic compound synthesis for converting energy of light of a specific wavelength emitted from said optical illuminator to heat.

13. The organic compound synthesizer according to claim 3, wherein said substrate allows light within a specified wavelength to pass through.

14. The organic compound synthesizer according to claim 3, wherein the optical illuminator includes a plurality of light sources.

15. The organic compound synthesizer according to claim 3, wherein said optical illuminator includes a light emitting diode light source and a lens having a focal length of about 4 mm.

16. The organic compound synthesizer according to claim 5, wherein said reaction liquid containing compounds necessary for synthesizing organic compounds and said reaction liquid containing a thermal acid generator are held in a space defined between said heat production substrate and said substrate for organic compound synthesis.

17. The organic compound synthesizer according to claim 5, wherein the specific wavelength of light emitted from the optical illuminator is set in the near infrared range or infrared range.

18. The organic compound synthesizer according to claim 1, wherein the substrate for organic compound synthesis is indirectly heated by the substrate heater.

19. The organic compound synthesizer according to claim 18, further comprising a drum and a thermal head, wherein the reaction liquid is supplied on the drum, the thermal head heats the reaction liquid on a desired region of the drum, and the heated reaction liquid is transferred onto the substrate.

20. The organic compound synthesizer according to claim 18, further comprising a transfer drum for receiving the heated reaction liquid from the drum and transferring it to the substrate.

21. A method for synthesizing organic compounds comprising:
providing a substrate for organic compound synthesis;
supplying a reaction liquid containing a thermal acid generator to said substrate for organic compound synthesis;
selectively heating a specific portion of said substrate for organic compound synthesis to thereby heat said reaction liquid containing a thermal acid generator for generating protons; and
supplying a reaction liquid containing compounds necessary for synthesizing organic compounds and containing at least one type of polymerizable repeat unit to said heated substrate for organic compound synthesis.

22. The method of claim 20, wherein the supplying of a reaction liquid containing compounds necessary for synthesizing organic compounds and containing at least one type of polymerizable repeat unit and the supplying of a reaction liquid containing a thermal acid generator to said substrate for organic compound synthesis comprise supplying a single reaction liquid containing both the compounds necessary for synthesizing organic compounds and containing at least one type of polymerizable unit and the thermal acid generator.

23. A reaction container for synthesizing organic compounds, comprising:
a substrate for organic compound synthesis including a top surface and a bottom surface;
a heat production substrate positioned a predetermined distance apart from the substrate for organic compound synthesis and including a bottom surface and a top surface opposite the bottom surface of the substrate for organic compound synthesis; and
a cover substrate that covers the top surface of the heat production substrate to thereby enclose the substrate for organic compound synthesis in a manner that defines a reaction liquid holding section around the substrate for organic compound synthesis, wherein
the heat production substrate is configured to heat a reaction liquid including a thermal acid generator in the reaction liquid holding chamber to create an acid region between the bottom surface of the substrate for organic compound synthesis and the top surface of the heat production substrate for use in organic compound synthesis.

24. The reaction container for synthesizing organic compounds according to claim 23, wherein the heat production substrate includes a quartz glass substrate having a thin molybdenum film on the top surface thereof.

25. The reaction container for synthesizing organic compounds according to claim 23, wherein the cover substrate also includes a reaction liquid inlet and a reaction liquid outlet that, together with the reaction liquid holding chamber, define a passage for reaction liquids.
